(19) United States
(12) Patent Application Publication
(10) Pub. No.: US 2018/0099151 A1
(43) Pub. Date: Apr. 12, 2018

(54) Method of Visualizing and Setting Dose Constraints for Radiation Therapy

(71) Applicant: Mitsubishi Electric Research Laboratories, Inc., Cambridge, MA (US)

(72) Inventors: Alan Sullivan, Middleton, MA (US); Teng-Yok Lee, Cambridge, MA (US)

(73) Assignee: Mitsubishi Electric Research Laboratories, Inc., Cambridge, MA (US)

(21) Appl. No.: 15/290,114

(22) Filed: Oct. 11, 2016

(51) Int. Cl. A61N 5/10; A61B 6/03

(52) U.S. Cl. CPC A61N 5/1031; A61N 5/1039; A61B 6/032

(57) ABSTRACT

Systems and methods for radiation treatment for determining a dose of radiation. Determining, for a tumor voxel, a value for maximum and minimum constraints, for treating the tumor voxel based on a distance function of a distance field of the tumor and distance fields of organs at risk (OARs). Each constraint on each tumor voxel is a function of a distance from the tumor voxel to a boundary of the tumor, and distances to boundaries of OARs. Determining, for an OAR voxel, a value of a maximum constraint based on the distance field of the tumor. Each constraint on each OAR voxel is a function of a distance from the OAR voxel to the boundary of the tumor. Determining a tumor constraint and a corresponding OAR constraint according to a threshold constraint set, to obtain the constraint set. Determine the dose of radiation, according to the constraint set.

### FIELD

The present disclosure relates generally to controlling radiation treatments, and more particularly systems and methods to controlling the operation of the treatment delivery system.

### BACKGROUND

Conventional radiation therapies of tumors are structured to accomplish two objectives, first removing of the tumor, and second preventing of damage to healthy tissue and organs-at-risk (OAR) near the tumor. Most tumors can be removed completely if an appropriate radiation dose is delivered to the tumor. Unfortunately, delivering certain amounts of doses of radiation to eliminate a tumor can likely result in complications, due to damaging healthy tissue and OAR's surrounding the tumor. One conventional technique to address this problem is a three-dimensional (3D) conformal radiation therapy that uses beams of radiation in treatment shaped to match the tumor to confine the delivered radiation dose to only the tumor volume defined by the outer surfaces of the tumor, while minimizing the dose of radiation to surrounding healthy tissue or adjacent healthy organs.

Typically, to perform these radiation treatment therapy plans, involves defining an exact type, locations, distribution and intensity of radiation sources so as to deliver a desired spatial radiation distribution. Radiation treatment therapy planning begins with a set of Computed Tomography (CT) images of a patient's body in the region of the tumor, i.e. CT images.

During the planning of the radiation treatment therapy the spatial distribution of radiation can be determined, usually by simulation, in terms of the dose of radiation, i.e. number of grays of radiation, that is deposited in each CT data voxels. The quality of planning radiation distribution is evaluated by comparing it to the desired plan goals, i.e. high radiation dose to the tumor and low radiation dose to the OARs. For example, a high quality plan can be one that achieves all of the desired plan goals, while a lower quality plan will fail to achieve some or all of the plan goals, for instance, by having a radiation dose delivered to a portion of an OAR that is in excess of the plan goals.

It is not uncommon to have an inherent conflict between the desire to have high dose in the tumor and low dose in a nearby OAR. However, certain OAR types may be redundant in their function and substantial portions of the OAR volume can be completely removed while retaining their function. Other OAR types lose their function if any of the structure is completely removed. For example, OARs may be an optic nerve or a brain stem, whose damage or destruction by radiation would be highly detrimental to the person undergoing therapy. Therefore, depending upon the radiation sensitivity of the OAR, the more sensitive OAR volumes that receive a measured dose of radiation, essentially depends upon no portion of the OAR being subjected to a lethal dose.

Conventional radiation treatment planning fail during the planning process, in part, because due to the tumor volume and/or the OAR volume are irregularly shaped having irregular spatial configurations with concave/contoured boarders, which result in the radiation beam being successful only part of the time. For example, because of the irregularly tumor shape, the relative arrangement of the tumor within 3D space can have twists or outer surfaces pointing inward, relative to a plane parallel to the path of the radiation beam, such that healthy tissue or OARs can be disposed approximate the concavities formed by the outer tumor concave surfaces. Specifically, given that the OAR anatomic structure is non-uniform, some important parts of OAR may be overdosed. Another problem with the planning process of conventional radiation treatment planning is that the user is not provided an explicit local control of trade-offs, i.e. parts of the OAR that are over exposed.

### SUMMARY

Some embodiments of present disclosure are based on a realization that when the dose optimization is infeasible there is no need to directly approximate all the constraints for either the tumor or the Organ-At-Risk (OAR), to obtain a dose constraint set. Instead, it is advantageous to efficiently determine the dose constraints that will be in conflict, notably, it is necessary to identify the subset of voxels that are in conflict.

Specifically, when a dose optimization is infeasible it is necessary to adjust one or more constraints in either the tumor or the OAR to obtain a constraint set, which may or may not result in a feasible constraint set. The effect of adjusting constraints is to allow some tumor voxels to receive a dose below the desired minimum dose, or to allow some OAR voxels to receive a dose above the desired maximum dose. These constraints are used as part of a technique in determining dose optimization, that the total dose is minimized subject to a set of constraints on some of the CT voxels. When regarding tumor voxels, it is necessary to constrain the dose to lie within an acceptable range of values, while for OAR voxels, the dose is constrained to lie below a maximum value. In other words, we observed that only some of these constraints will be in conflict, the constraints on tumor voxels far from an OAR can be satisfied with no effect on OAR dose, as can constraints on OAR voxels far from tumor voxels.

Having understood only some of these constraints will be in conflict, we further realized it is necessary to identify and potentially visualize a representation of the subset of voxels in conflict, for assisting in evaluating and controlling the tradeoff between constraints that are in conflict. Because constraints are interdependent, change in one constraint can effect another one. We recognized the dependency among the constraints is a function of distances between the voxels corresponding to those constraints.

Embodiments of present disclosure are based on a realization that a distance between the tumor and OAR can be a single parameter of optimization of the dose of radiation. Specifically, our realization is that constraints within the tumor and OARs can be set using so-called “distance fields” of the tumor and OAR.

For example, a value of a distance field of an object, e.g., a tumor and/or OAR, at a point in space (p) can be a Euclidean distance, or shortest distance, from the point (p) to a boundary of the object. Points that are on the boundary of the object have distance field values of zero. Wherein, the distance field is “signed” such that for point within the boundary of the object the distance field has one sign, for example positive, and outside the boundary the distance field has the opposite sign, for example negative. For each object, i.e. tumor and/or OAR, there is a unique distance field that is determined by the object's shape, in particular, the shape of the object's boundary. In other words, the unique distance field is specific to the shape of the object's boundary, so as to create isocontours (curves of constant distance) for the object, i.e. tumor and/or OAR. Therefore, it is useful to have methods and systems to assign initial constraint values that take into account the non-uniform sensitivity of the tumor to radiation. At least one aspect to using the tumor's distance field, is that it becomes possible to compensate for the biological effects of the tumor's non-uniformity by determining the initial dose constraints for the tumor voxels through a mathematical function of the distance field of the tumor. Essentially, the present disclosure provides for methods and systems to identify and control the constraints specific to the tumor and the OARs, and control a tradeoff between constraints that are in conflict.

Another realization the present disclosure is based upon is that the dose constraints of the tumor voxels and the OARs voxels can be determined as a function of the distance fields both the tumor and the OARs, as well as of the radiation dose to the voxels of the tumor and OARs. The determining of the constraints using the value of the radiation dose is useful when dealing with infeasible dose optimization.

To better understand this realization, imagine transforming an analogy of the tumor being thermally hot into a display form, where the thermally hot tumor and the thermally affected healthy tissue and OARs could be visualized. We would then be able to visually identify the constraints in conflict merely by the varying degrees of the thermally affected areas. However, implementing this realization presented challenges, such that despite overcoming the challenge of how to identify and control the constraints distributed in a 3D space, we still needed to address how to actually visualize and control the set of conflicting constraints on the tumor and OARs in the 3D space.

We discovered that rather than trying to visualize and control the set of conflicting constraints in 3D space, it is much easier to do so in a one-dimensional (1D) space. Then, the 1D space could make it possible to graph the constraint values via the 1D coordinate, so as to identify the set of constraints in conflict and to affect a modification. Using the Euclidean distance, i.e., the shortest distance from a given voxel to the boundary of the tumor or OAR, transforms the 3D space of the constraint values into a 1D space. By making it possible to graph the constraint values via the 1D coordinate system, we are able to visualize the constraint conflict. In other words, we plot the OAR constraints verses distance to a tumor boundary and tumor constraints verses the distance to an OAR boundary on the same graph. After plotting the OAR constraints and the tumor constraints on the same graph, we are able to construct a user interface, i.e. a slider or control point, that causes a shift of the characteristic of the curve along the distance axis and a corresponding localized change to the tumor and OAR constraints.

In particular, we are able to construct systems and methods that are able to make minimal adjustments to the constraints as necessary to obtain a feasible optimization problem, as well as, generate a graphical representation illustrating simultaneously the constraints in conflict. Specifically, we are able to simultaneously illustrate visually, the effects of the specific dose of radiation to the tumor, along with the corresponding damaging effects to the surrounding healthy tissue and OARs for the specific radiation dose. By providing a visual display to the user, the user now has the ability to have explicit local control of constraint trade-offs, i.e., parts of OAR that are overdosed via the slider. For example, the user could be a dosimetrist, doctor or person associated with determining dosing or medical issues for the patient. Wherein the user, via the slider of the present disclosure, will have the ability to visually see each potential radiation dose to the tumor and the corresponding damaging effects to the OARs, and after having reviewed all the possible radiation dosing options, make an informed dosing radiation decision necessary to obtain a feasible constraint set or specific dose of radiation for the patient. Essentially, the present disclosure provides for the user or doctor to not only be able to identify and control the constraints specific to the tumor and the OARs, but just as importantly, also be able to graphically visualize controlling a tradeoff between constraints that are in conflict. In part, the slider in combination with the features of the present disclosure is able to provide the doctor with informed radiation dosing decisions, as well as have provide for an increased accuracy in understanding how the proposed dosing of radiation will have on the tumor and OARs of the patient.

According to an embodiment of the present disclosure, a radiation treatment method for determining a dose of radiation. The method including determining, for a tumor voxel in a set of tumor voxels from stored data, a value of a maximum dose constraint and a minimum dose constraint, for treating the tumor voxel based on a distance function of at least one distance field of the tumor and distance fields of organs at risk (OARs). Wherein each dose constraint on each tumor voxel is a function of a distance from the tumor voxel to a boundary of the tumor, and distances to boundaries of OARs. Determining, for an OAR voxel in a set of OAR voxels in an OAR of interest of the OARs, a value of a maximum dose constraint based on the at least one distance field of the tumor. Wherein each dose constraint on each OAR voxel is a function of a distance from the OAR voxel to the boundary of the tumor. Determining a tumor dose constraint and a corresponding OAR dose constraint to obtain a dose constraint set according to a threshold dose constraint set. Determine the dose of radiation, according to the dose constraint set, wherein the dose of radiation is used for managing the radiation treatment planning system.

According to another embodiment of the present disclosure, a radiation treatment planning system for determining a dose of radiation. The system including at least one processor and at least one non-transitory storage memory having stored data and computer readable instructions executable by the at least one processor. Wherein the execution of the computer readable instructions by the at least one processor is configured to determine, for a tumor voxel in a set of tumor voxels from patient imaging data of the stored data, a value of a maximum dose constraint and a minimum dose constraint, for treating the tumor voxel based on a distance function of at least one distance field of the tumor and distance fields of organs at risk (OARs) in a set of OARs. Wherein each dose constraint on each tumor voxel is a function of a distance from the tumor voxel to a boundary of the tumor, and distances to boundaries of OARs in the set of OARs. Determine, for an OAR voxel in a set of OAR voxels in an OAR of interest from the set of OARs, a value of a maximum dose constraint based on the at least one distance field of the tumor. Wherein each dose constraint on each OAR voxel is a function of a distance from the OAR voxel to the boundary of the tumor. Determining a tumor dose constraint and a corresponding OAR dose constraint to obtain a dose constraint set according to a threshold dose constraint set. Determine the dose of radiation, according to the dose constraint set, wherein the dose of radiation is used for managing the radiation treatment planning system.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1A is a block diagram illustrating a method for radiation treatment for determining a dose of radiation, according to embodiments of the present disclosure;

FIG. 1B is a schematic illustrating the method of FIG. 1A, implemented using a diagnostic system and radiation treatment system to a patient's body, according to embodiments of the present disclosure;

FIG. 1C is a block diagram illustrating the method of FIG. 1A, that can be implemented in a radiation therapy treatment planning system for determining a dose of radiation, according to embodiments of the present disclosure;

FIG. 1D is a block diagram of illustrating the method of FIG. 1A, that can be implemented using an alternate computer, according to embodiments of the present disclosure;

FIG. 2A and FIG. 2B are schematics illustrating the concept of distances, FIG. 2A shows isocontours (curves of constant distance) for the tumor, and FIG. 2B shows the same for the OAR, according to embodiments of the present disclosure;

FIG. 2C shows isocontours of the distance field of the tumor inside of the tumor's boundary, according to embodiments of the present disclosure;

FIG. 3A is a block diagram illustrating a method that includes method of FIGS. 1A-1C, in addition to converting the dose of radiation into a curve of irradiation in the body of the patient as a function of a distance to a border of the tumor, a border of the OAR, or both, to generate a graphical representation to identify the dose constraints in violation, according to embodiments of the present disclosure;

FIG. 3B is a schematic illustrating the method of FIG. 3A that includes using the processor to convert the dose of radiation into the curve of irradiation in the body of the patient via a user shifting over the distance between the tumor and OAR on a graphical display, according to embodiments of the present disclosure;

FIG. 3C is a block diagram illustrating a method that initially converts an acquired initial dose of radiation into a curve of irradiation in the body of the patient as a function of the distance to the boundary of the tumor, the boundary of the OAR, or both, to generate a graphical representation to identify the dose constraints in violation, according to embodiments of the present disclosure;

FIG. 4 is a graph illustrating the tumor and OAR constraints modified, i.e. increased in the OAR constraint and decreased in the tumor constraint, as a consequence of the user's or dosimetrist's action in setting the location of the control point at a point intermediate between the two constraint sets, according to embodiments of the present disclosure;

FIG. 5 is a schematic illustrating a set of dose fall-off curves that can be obtained by sampling a computed radiation dose from a set of points on the OAR along a set of distance sampling vectors, according to embodiments of the present disclosure;

FIG. 6 is a graph illustrating an overlap volume histogram (OVH) of the OAR indicating a fraction of the OAR voxels within a given distance of the tumor, according to embodiments of the present disclosure.

FIG. 7 is a graph illustrating a long dashed box on the left showing the tumor minimum and maximum dose constraints, the dot-dashed box on the right showing the OAR maximum dose constraints, and curves A and B that are two characteristic dose curves whose fall-off is the fastest achievable due to limitations in the beam size and tissue heterogeneity, according to embodiments of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. Contemplated are various changes that may be made in the function and arrangement of elements without departing from the spirit and scope of the subject matter disclosed as set forth in the appended claims.

Specific details are given in the following description to provide a thorough understanding of the embodiments. However, understood by one of ordinary skill in the art can be that the embodiments may be practiced without these specific details. For example, systems, processes, and other elements in the subject matter disclosed may be shown as components in block diagram form in order not to obscure the embodiments in unnecessary detail. In other instances, well-known processes, structures, and techniques may be shown without unnecessary detail in order to avoid obscuring the embodiments. Further, like reference numbers and designations in the various drawings indicated like elements.

Also, individual embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process may be terminated when its operations are completed, but may have additional steps not discussed or included in a figure. Furthermore, not all operations in any particularly described process may occur in all embodiments. A process may correspond to a method, a function, a procedure, a subroutine, a subprogram, etc. When a process corresponds to a function, the function's termination can correspond to a return of the function to the calling function or the main function.

Furthermore, embodiments of the subject matter disclosed may be implemented, at least in part, either manually or automatically. Manual or automatic implementations may be executed, or at least assisted, through the use of machines, hardware, software, firmware, middleware, microcode, hardware description languages, or any combination thereof. When implemented in software, firmware, middleware or microcode, the program code or code segments to perform the necessary tasks may be stored in a machine readable medium. A processor(s) may perform the necessary tasks.

### Definition of Terms

According to the definition of terms with regard to the present disclosure, the term “Radiation therapy” is considered the treatment of medical diseases by the application of ionizing radiation, for example ion beams, alpha emitters, x-rays or gamma rays. An example application of radiation therapy is the treatment of cancer using beams of ions, such as protons or carbon ions, such that the cancer cells are killed by the dose of radiation while adjacent healthy tissue is spared.

Further, the term “radiation therapy planning” begins with a set of Computed Tomography (CT) images of the patient's body in the region of the tumor, hereafter referred to CT images. The set of CT images is composed of a plurality of 2D cross-section images of the body oriented in a plane nominally perpendicular to the spine, with each successive image in the set of CT images corresponding to successive adjacent positions along the axis of the body. Each picture element (pixel) in a single CT image corresponds to the amount of x-rays absorbed by the tissue at that 3D spatial location. Radiation therapy planning involves a specification of at least two categories of spatial regions within the patient's CT data: a first category, often called tumor, is where a high and possibly uniform radiation dose is desired, and a second category, often called organ-at-risk (OAR), is where it is desired that the radiation dose be as low as possible. The specification of a type for the CT data voxels can either be a tumor voxel or OAR voxel, that is commonly represented as one of the following: a second 3D array of voxel data, the segmentation voxel data, or co-located with the CT voxel data. Wherein, each voxel of the segmentation voxel data has a label, e.g. an integer, whose value indicates the type of the corresponding voxel in the CT voxel data.

The term “dose of radiation” can be understood that the amount radiation deposited in a voxel of the CT voxel data is called the dose of radiation. Dose is a well-defined physical quantity given in units called Gray, where 1. Gray corresponds to 1 joule of radiation energy deposited in 1 kilogram of tissue.

The term organ-at-risk (OAR) can be understood that each OAR may have a different sensitivity to radiation, or a different level of importance in comparison to other OARs, such that each may have a different maximum permitted dose.

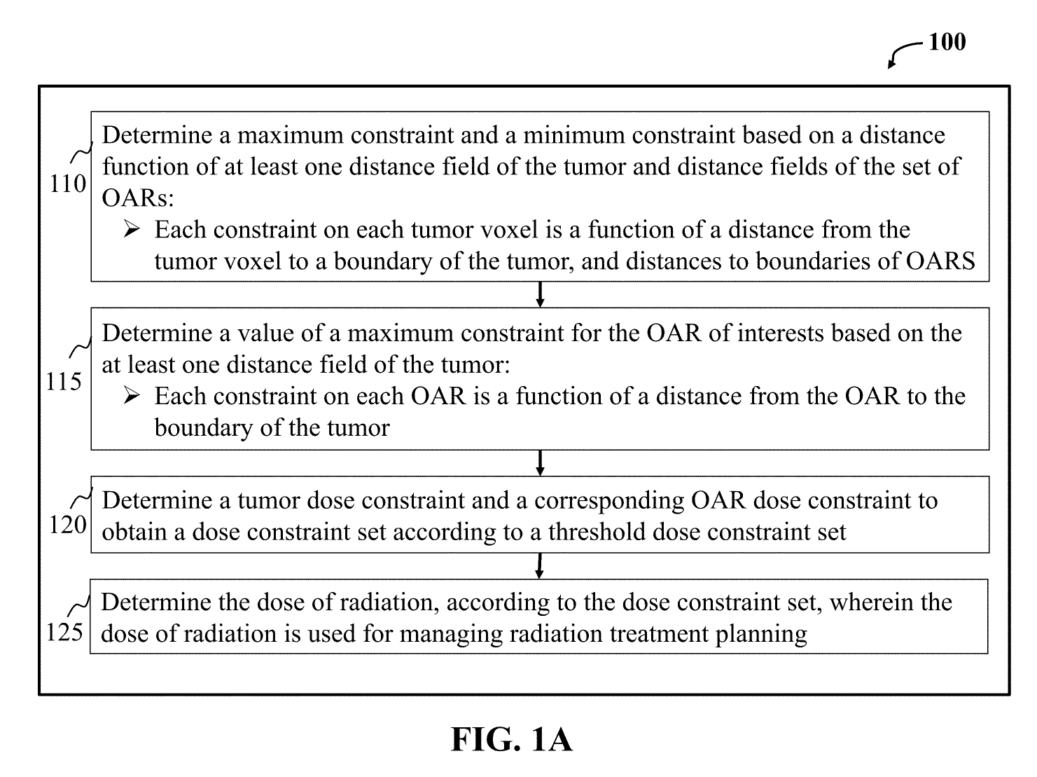

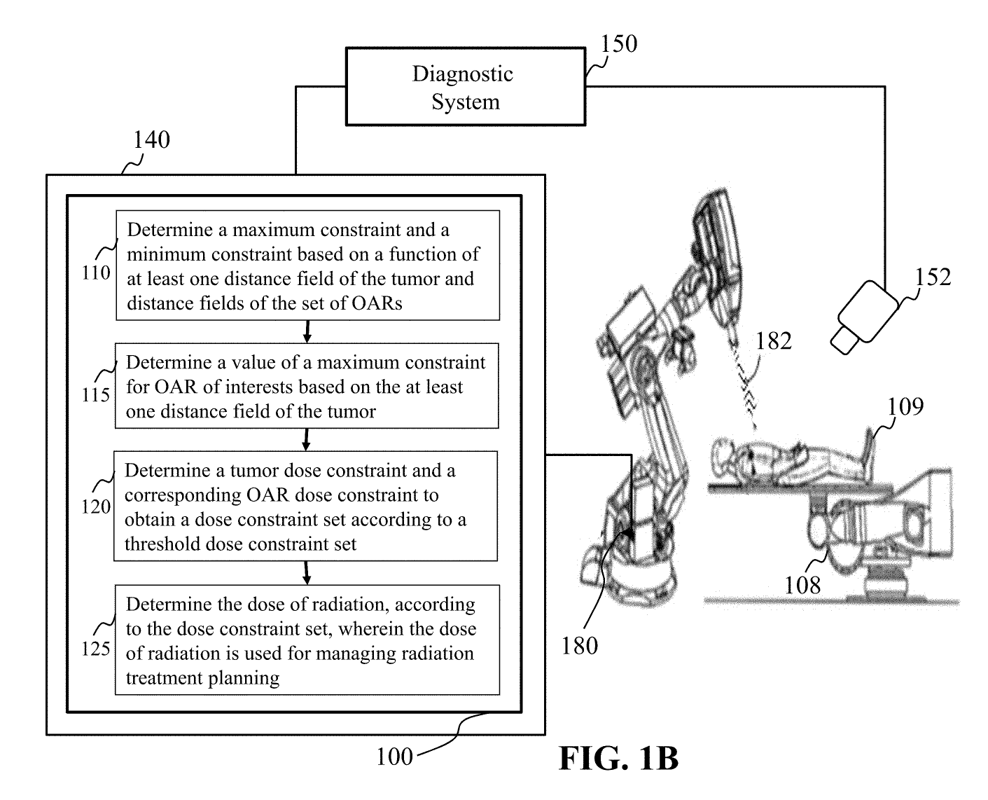

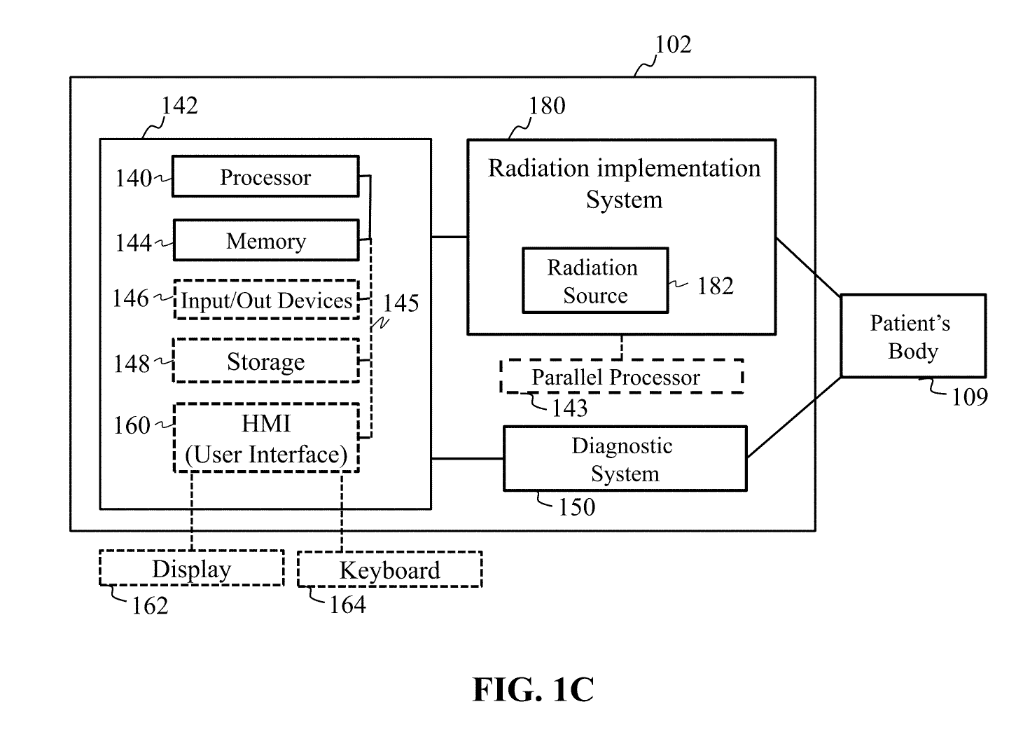

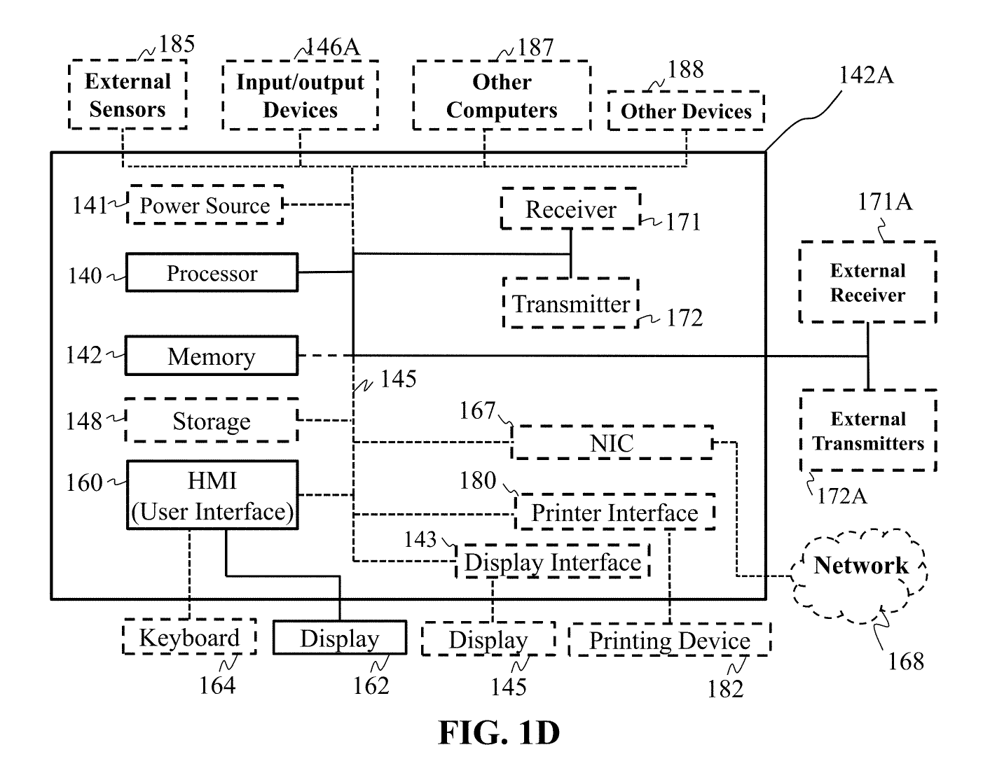

FIG. 1A is a block diagram illustrating a method 100 for radiation treatment for determining a dose of radiation, according to embodiments of the present disclosure. The method 100 including the step 110 determining for a tumor voxel in a set of tumor voxels from stored data, a value of a maximum dose constraint and a minimum dose constraint, for treating the tumor voxel. The stored data includes a set of Computed Tomography (CT) images, of a patient's body in the region of the tumor, such that pixel data of the CT images can be combined together into a single three-dimensional (3D) array of volume data elements, i.e. tumor voxels and organ-at-risk (OAR) voxels.

The constraints are used as part of a technique in determining dose optimization, such that the total dose is minimized subject to a set of constraints on some of the CT voxels. When regarding tumor voxels, it is necessary to constrain the dose to lie within an acceptable range of values, while for OAR voxels, the dose is constrained to lie below a maximum value. Specifically, when a dose optimization is infeasible it is necessary to adjust one or more constraints in either the tumor voxel or the OAR voxel to obtain a constraint set that could later be determined as a feasible constraint set. The effect of adjusting constraints is to allow some tumor voxels to receive a dose below the desired minimum dose, or to allow some OAR voxels to receive a dose above the desired maximum dose. The constraints are interdependent, such that change in one constraint can effect another one. Dependency among the constraints is a function of distances between the voxels corresponding to those constraints.

Still referring to Step 110 of FIG. 1A, the method is based on a realization that it is advantageous to efficiently determine the constraints that will be in conflict, notably, it is necessary to identify the subset of voxels that are in conflict. To better understand this realization, imagine the tumor is thermally hot, where the thermally hot tumor affects healthy tissue and OARs near the hot tumor. We would then be able to identify the constraints in conflict merely by the varying degrees of the thermally affected areas. Essentially, the present disclosure provides for identifying and controlling the constraints specific to the tumor and the OARs, by using a distance function of at least one distance field of the tumor and distance fields of organs at risk (OARs). The term distance field can be understood as Euclidean distances, from the boundaries of the tumor and OARs. In other words, our realization included the insight that constraints within the tumor and OARs can be set using “distance fields” of the tumor and OAR, i.e. using a distance function of at least one distance field of the tumor and distance fields of organs at risk (OARs). For example, as noted above, a value of a distance field of an object, e.g., a tumor, at a point in space (p) is the Euclidean distance, or shortest distance, from the point (p) to the boundary of the object. Points that are on the boundary of the object have distance field values of zero. Additionally, the distance field is “signed” such that for point within the boundary of the object the distance field has one sign, for example positive, and outside the boundary the distance field has the opposite sign, for example negative. Additionally, the distance fields of a tumor and a set of OARs may also be combined with a computed dose distribution to form a user interface for visualization and control of constraint tradeoffs between the tumor and OARs.

Continuing with step 110 of FIG. 1A, the method determines the value of the maximum dose constraint and the minimum dose constraint for the tumor voxel based on a distance function of at least one distance field of the tumor and distance fields of organs at risk (OARs). For example, the distance function can be a linear combination algorithm or a non-linear combination algorithm, of a tumor distance field and the OAR distance field. Wherein each dose constraint on each tumor voxel is a function of a distance from the tumor voxel to a boundary of the tumor, and distances to boundaries of OARs. For example, the subsequent tradeoffs between tumor and OAR likely do depend on the dose of radiation.

Step 115 of FIG. 1A, determines for an OAR voxel in a set of OAR voxels in an OAR of interest of the OARs, a value of a maximum dose constraint based on the at least one distance field of the tumor. Wherein each dose constraint on each OAR voxel is a function of a distance from the OAR voxel to the boundary of the tumor. For example, the subsequent tradeoffs between tumor and OAR likely do depend on the dose of radiation.

Step 120 of FIG. 1A, determines a tumor dose constraint and a corresponding OAR dose constraint to obtain a dose constraint set, according to the threshold dose constraint set. For example, the threshold dose constraint set may include a user selected threshold dose constraint set. At least aspect of utilizing a user selected threshold dose constraint set, is because it is often necessary to design a compromise between the desire for high radiation dose to the tumor and low radiation doses to the OARs. The exact nature of the compromise depends on the desires of the doctor and patient and are not necessarily mathematical in nature. Some patients may choose, for example, to be blinded so that the tumor is completely eliminated. While others may instead prefer to retain their sight by accepting some reduced quality of tumor control. Additionally, some OARs, such as the liver, are such that part of them can be destroyed with radiation without a total loss of function, whereas others, such as the optic nerve, suffer a total loss of functionality if any part is damaged by radiation.

For example, it is possible for the doctor/user could be a dosimetrist, the dosimetrist selects the threshold dose constraint set based on making a compromise between: (1) sparing the OAR, but under dosing the tumor; (2) adequately irradiating the tumor, but overdosing some or all of an OAR; or (3) some tradeoff between the two. Further, the user selected threshold dose constraint set can also be based on, patient records, statistical models that are used to simplify creating new radiation treatment plans including dose of radiation treatments, as well as prior radiation treatment plans and/or experiences gained from each prior radiation treatment plan.

The user selected threshold dose constraint set may also be based on, one or a combination of: the determined dose constraints, i.e. the maximum and the minimum dose constraints for the tumor voxel and the maximum dose constraint for the OAR voxel; a compromise between an amount of tumor control of a specific tumor dose constraint versus an amount of damage control to an OAR voxel of a specific corresponding OAR dose constraint; stored historical radiation dose treatments for tumor types, tumor voxels and OARS similar to the set of tumor voxels and OARs from the stored data, among other things.

Step 125 of FIG. 1A, determines the dose of radiation, according to the dose constraint set, wherein the dose of radiation is used for managing the radiation treatment planning system. The radiation treatment planning system may further include

FIG. 1B is a schematic illustrating the method of FIG. 1A, that can be implemented using a diagnostic system and radiation treatment system to a patient's body, according to embodiments of the present disclosure. The method 100 can be executed in at least one processor 140 and is in communication with a diagnostic system 150. The diagnostic system 150, is in communication with a radiation implementation system 180, and generates empirical data of a patient or body 109, i.e., a body of a human or other living thing, positioned on table 108. The diagnostic system 150 can include sensors contacting the body 109 or not contacting the body 109, to obtain the empirical data of body 109. An example diagnostic system 150 may include a sensor 152 that is a camera, a computed tomography (CT) scanner, a magnetic resonance imaging (MRI) scanner, a positron emission tomography (PET) scanner, or the like. The empirical data may be used as input information to the radiation implementation system 180, to implement the dose of radiation determined from the at least one processor 140.

The radiation treatment system 180 can include a radiation source 182 that emits a directed beam of radiation for treatment to the body 109. Examples of radiation sources may include, an X-ray source, a gamma ray source, an electron beam source, etc. The radiation source 182 may further comprise a multi-leaf collimator (MLC) to shape the beam. By adjusting the position of the leaves of the MLC, a radiotherapist can match the radiation field to a shape of the treatment volume of body. Other beam shaping and/or contouring can be included in some embodiments. The radiation source 182 can have a corresponding source model. The radiation system 180 may be controlled by the radiation treatment planning method 100, for example, to deliver intensity modulated radiation energy and to conform radiation treatment to the shape of the intended radiation treatment volume.

FIG. 1C is a block diagram illustrating the method of FIG. 1A and FIG. 1B, that can be implemented in a radiation therapy treatment planning system 102 for determining a dose of radiation, according to embodiments of the present disclosure. The radiation therapy treatment planning system 102 includes a computer 142, a radiation implementation system 180, a diagnostic system 150 and a patient's body 109.

The computer 142 of FIG. 1C, includes the processor 140 and memory 144 connected through a bus 145. The processor 140 can be configured to execute stored instructions, as well as be in communication with a memory 122 that stores instructions that are executable by the processor 140. The processor 140 can be a single core processor, a multi-core processor, a computing cluster, or any number of other configurations. The memory 142 can include random access memory (RAM), read only memory (ROM), flash memory, or any other suitable memory systems.

Optionally, the computer 142 of FIG. 1C, can include input/output devices 146 and storage device 148. The input/output device 109 may include, for example, a mouse, a keyboard, an interface for data transfer over a network or a data bus. The computer 142 can include a storage device 148 adapted to store supplementary data and/or software modules that can be used by processor 140. For example, the storage device 148 can store historical medical data relating to similar different types of tumors, patient and historical radiation treatment data, related to for example, the patient imaging data from the stored data. The storage device 148 can include a hard drive, an optical drive, a thumb-drive, an array of drives, or any combinations thereof.

The computer 142 of FIG. 1C can include a Human Medium Interface (HMI), i.e. user interface that includes a display device 162 and keyboard 164, which all can be connected through bus 145. It is possible the display device 162 can include a computer monitor, camera, television, projector, or mobile device, and the like. The display device 162 may also be, for example, a liquid crystal display (LCD), a cathode ray tube (CRT) monitor, a plasma display, etc.

Still referring to FIG. 1C, the radiation implementation system 180 is similar to that of FIG. 1B, including the radiation source 182 that emits the directed beam of radiation for treatment to the body 109. It is contemplated empirical data from the diagnostic system 150 may also be used as input information to the radiation implementation system 180, such that a parallel processor 143 may be used to determine an alternate dose of radiation, different from the determined dose of radiation from the at least one processor 140. It is noted that the result is independent of processor architecture. This aspect also applies equally well to serial and parallel processors. The parallel processor can also be adapted to receive input information concerning the body 109 having an intended radiation treatment volume that can be represented as a volume of voxels. The parallel processor 102 can also be adapted to generate output information for providing radiation treatment to the intended radiation treatment volume of the body.

FIG. 1D is a block diagram illustrating the method of FIG. 1A, FIG. 1B and FIG. 1C, using an alternate computer 142A, according to embodiments of the present disclosure. The computer 142A includes the processor 140, memory 144, storage 148 and user interface 160 with display 162 and keyboard 164, which are connected through bus 145.

The computer 142A can include a power source 141, depending upon the application the power source 141 may be optionally located outside of the computer 142A. Linked through bus 145 can be a display interface 143 adapted to connect to a display device 147, wherein the display device 147 can include a computer monitor, camera, television, projector, or mobile device, among others. A printer interface 180 can also be connected through bus 145 and adapted to connect to a printing device 182, wherein the printing device 182 can include a liquid inkjet printer, solid ink printer, large-scale commercial printer, thermal printer, UV printer, or dye-sublimation printer, among others. A network interface controller 167 is adapted to connect through the bus 145 to a network 168. Medical data or related data, among other things, can be rendered on a display device, imaging device, and/or printing device.

Still referring to FIG. 1D, the medical data or related data, among other things, can be transmitted over a communication channel of the network 168, and/or stored within the storage system 148 for storage and/or further processing. Further, the medical data or related data may be received wirelessly or hard wired from a receiver 171 (or external receiver 171A) or transmitted via a transmitter 172 (or external transmitter 172A) wirelessly or hard wired, the receiver and transmitter are both connected through the bus 145. The computer 142A may be connected to external sensors 185 and external input/output devices 146A. For example, the external sensors 185 may include sensors for, operating room conditions, patient related information, etc. The computer 142A may be connected to other external computers 187 or other devices 188, the other devices may be electronic information related devices, measurement related devices and other communication devices.

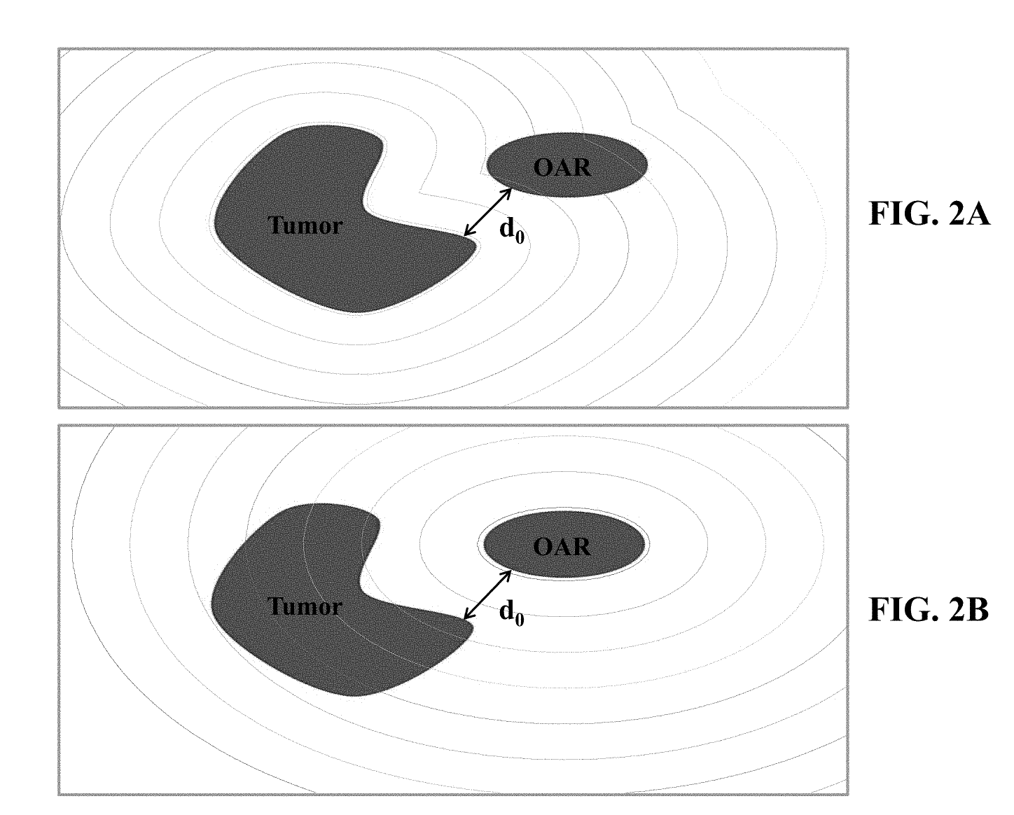

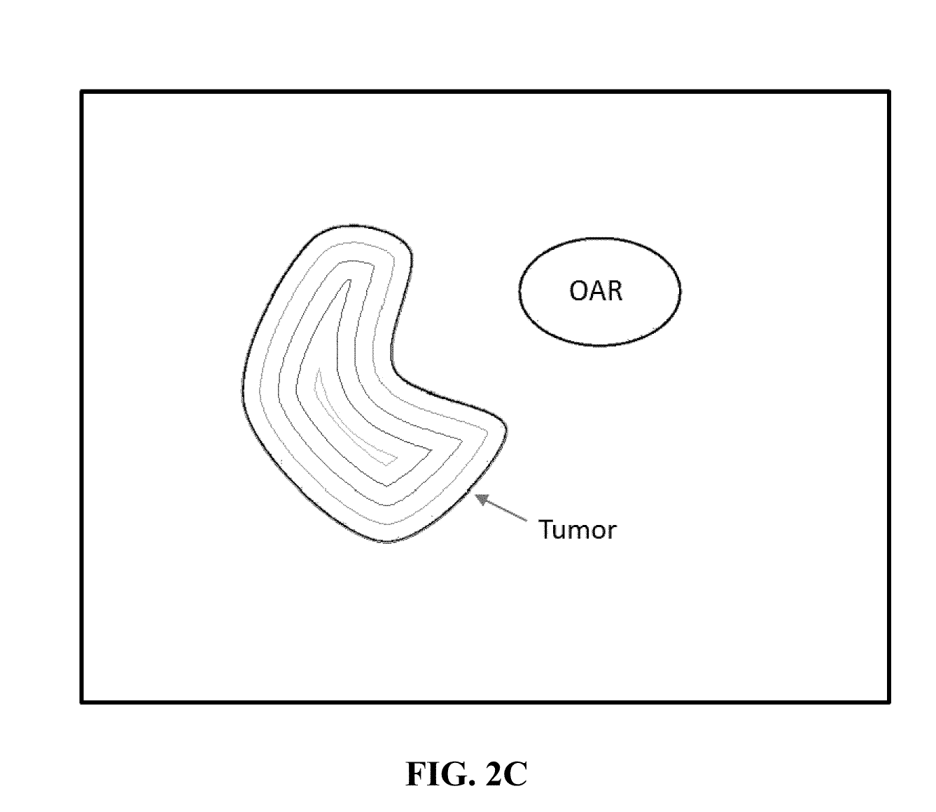

FIG. 2A and FIG. 2B are schematics illustrating the concept of distances between tumor and OARs, according to embodiments of the present disclosure. FIG. 2A shows isocontours (curves of constant distance) for the tumor, and FIG. 2B shows the same for the OAR. For each object there is a unique distance field that is determined by the object's shape, in particular the shape of the object's boundary. As noted above, it is necessary for the doctor to assign initial values to the maximum and minimum dose constraints for each tumor voxel. While it is common to assign a uniform value to all tumor voxels, a uniform value may not supply a sufficiently high dose to the interior of the tumor due to the biology of the tumor for example its low oxygen level in the tumor interior. Therefore, it is useful to have methods and systems that can assign initial constraint values that take account of the non-uniform sensitivity of the tumor to radiation.

Using the tumor's distance field, it becomes possible to compensate for the biological effects of the tumor's non-uniformity by determining the initial dose constraints for the tumor voxels through a mathematical function of the distance field of the tumor. For example, if we denote the tumor distance field as d_{T}(x, y, z) as the value of the tumor distance field at point (x, y, z), then we may choose that the tumor's initial minimum dose constraints are given by a linear mathematical function such as

D_{tumor,min}(x,y,z)=D_{T,min}+kd_{T}(x,y,z)

where D_{T, min} is the minimum tumor dose at the tumor boundary, for example 50 Gray, and k is a scale parameter that can be either positive or negative depending on whether the tumor dose constraint should increase or decrease, respectively, within the tumor interior.

FIG. 2C shows the isocontours of the distance field of the tumor inside of the tumor's boundary, according to embodiments of the present disclosure. Using the tumor's distance field to assign initial dose constraints according to a linear function will result in dose constraint isocontours that correspond to the distance field isocontours of FIG. 2C.

Alternately, the function can be non-linear. For example, the doctor may want to limit the maximum value that a tumor voxel's constraint can achieve to be ≦D_{tumor, min}^{max} to avoid other undesirable consequences of excessively high radiation dose. This can be achieved by using a min( ) function, where min(a, b) returns the lesser of the two input values, a or b. In this case our tumor constraint equation becomes

D_{tumor,min}(x,y,z)=min(D_{T,min}+kd_{T}(x,y,z),D_{tumor,min}^{max}).

This non-linear function will increase linearly as the value of the distance field increases with slope k from a value of D_{T, min} at the boundary of the tumor, but be limited to no more than D_{tumor, min}^{max}. There are many other possible non-linear functions, e.g., Gaussian, exponential, logarithm, that are known in the art and may be optionally selected by the doctor for some reason outside the scope of this invention.

Still referring to FIG. 2C, after the first dose optimization has been performed the quality of the resulting dose is evaluated as to whether each of the tumor and OAR constraints has been satisfied. It is likely that, for reasons described above, there will exist a subset of the set of tumor voxels and OAR voxels in which the dose is not within the range given by the initial constraints. For these subsets of voxels, it is desirable to be able to determine a compromise.

Therefore, an additional feature of the present disclosure is that the dose constraints of the tumor voxels and the OARs voxels can be determined as a function of the distance fields both the tumor and the OARs, as well as of the radiation dose to the voxels of the tumor and OARs. The determining of the constraints using the value of the radiation dose is useful when dealing with infeasible dose optimization.

Still referring to FIG. 2C, when a dose optimization is infeasible it may be necessary, but not strictly necessary, because the user can accept the infeasible results. However, it is desirable if possible to purposefully select the compromise so as to affect a beneficial tradeoff. to adjust one or more the dose constraints for either tumor voxels or OAR voxels to obtain a feasible constraint set. By adjust we mean that we might reduce the minimum dose constraint to a set of tumor voxels adjacent to an OAR so that the OAR receives a lower dose and satisfies the OAR's maximum dose constraints. Alternately, we might adjust the OAR maximum dose constraints by increasing these so that voxels within the tumor that are adjacent to the OAR receive a dose above the tumor voxel's minimum dose constraint. Finally, the adjustment may reduce some tumor constraint and increase some OAR constraints so as to split the constraint violation between both: some tumor voxels are under dosed, and some OAR voxels are overdosed.

Clearly only a subset of the set of tumor voxels and a subset of the set of OAR voxels need to be adjusted. We can select the sets of voxels and adjust their constraints by considering the distance fields of the tumor and the OARs as well as the dose of radiation that is computed using the initial constraints.

Upon an initial review of FIG. 2A, FIG. 2B and FIG. 2C, it is clear that those points on the OAR closest to the tumor will have a higher dose than those farther from the tumor. We realized that the 1D space could make it possible to graph the constraint values via the ID coordinate, to identify the set of constraints in conflict and to affect a modification. We determined that the ideal 1D coordinate is the signed Euclidean distance to the tumor or OAR boundaries. The Euclidean distance, i.e., the shortest distance from a given voxel to the boundary of the tumor or OAR, transforms the 3D space of the constraint values into a 1D space. For example, FIG. 2A shows the set of isocontours (curves of constant distance) from the boundary of the tumor, and FIG. 2B shows the same for the OAR, as noted above. The minimum distance, do, is indicated in both FIG. 2A and FIG. 2B. Wherein the voxels inside the tumor or OAR have negative distance values, while those outside the tumor or OAR have positive distance values. As noted above, to better understand this concept, it is useful to imagine that the tumor is thermally hot. In this analogy it becomes clear that the parts of any OAR that are close to the tumor will be heated more by the tumor, than those parts farther from the tumor. To visualize the constraint conflict, we plot OAR constraints verses distance to the tumor boundary and tumor constraints verses the distance to the OAR boundary on the same graph.

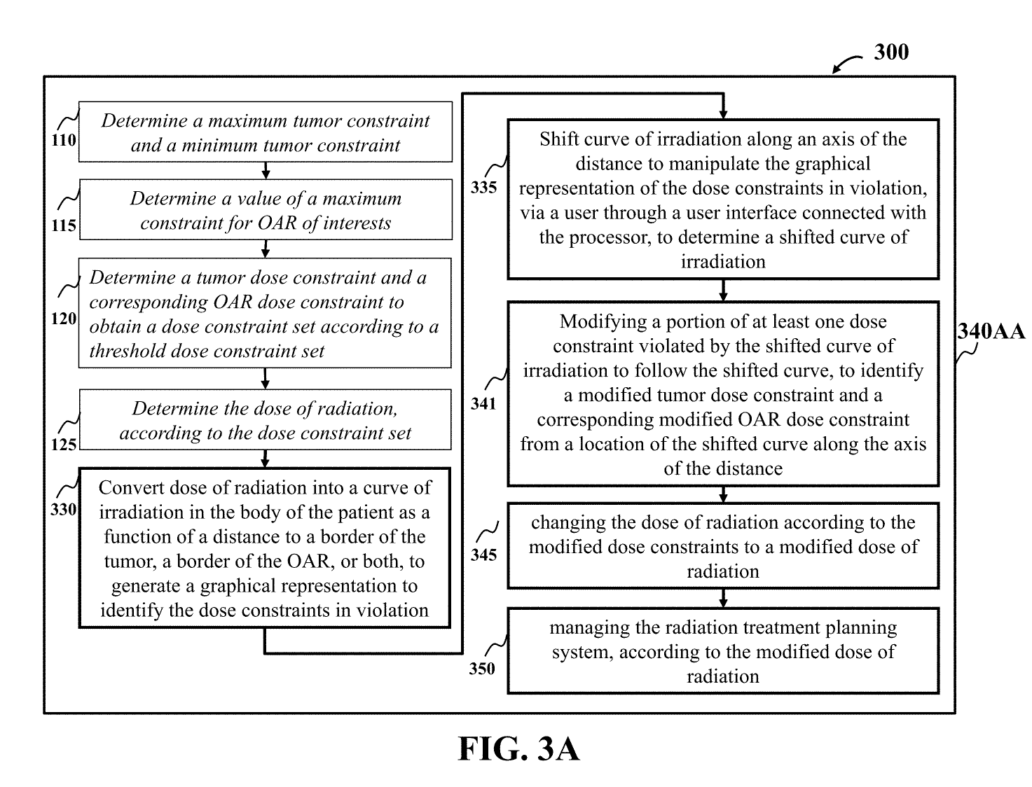

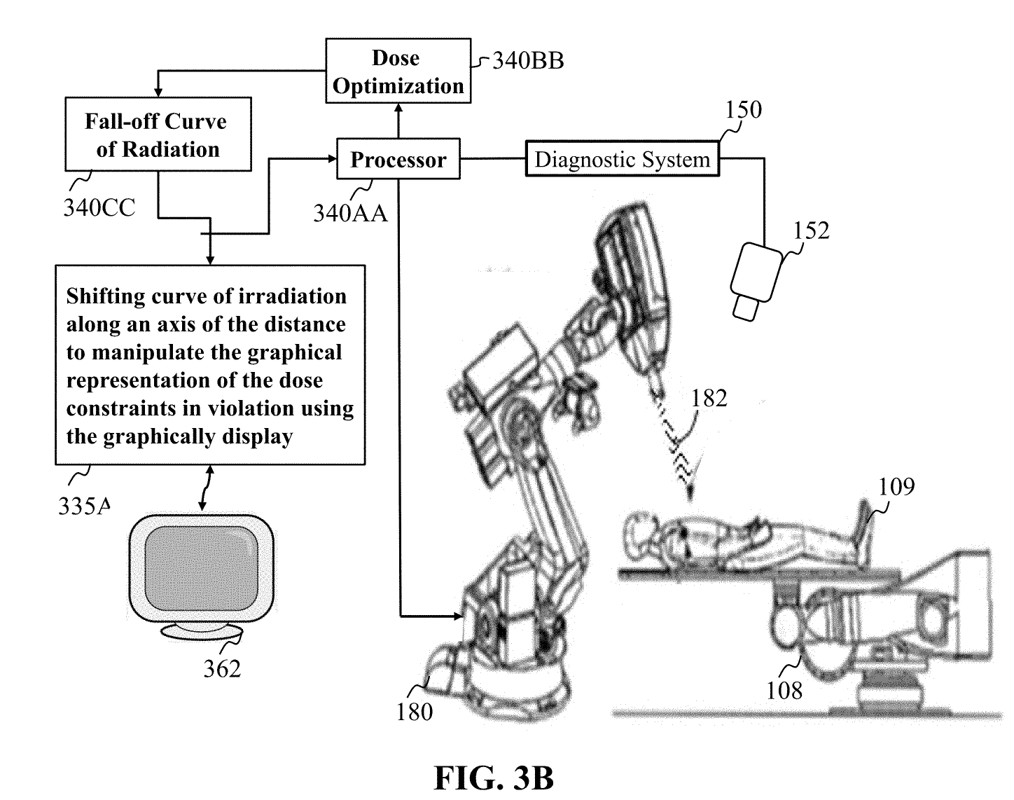

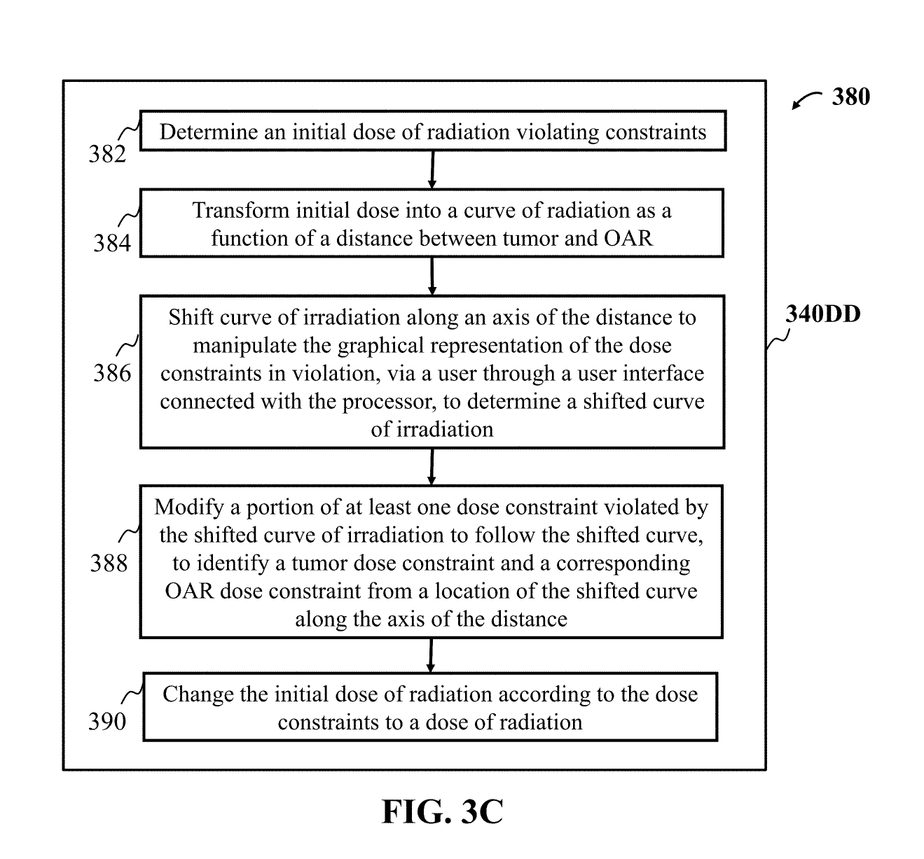

FIG. 3A is a block diagram illustrating a method 300 processed by processor 340AA that includes the method 100 of FIGS. 1A-1C, and in addition, is converting the dose of radiation into a curve of irradiation in the body of the patient as a function of a distance to a border of the tumor, a border of the OAR, or both, to generate a graphical representation to identify the dose constraints in violation, according to embodiments of the present disclosure.

Method 300 illustrates the realization of rather than trying to control the set of conflicting constraints in 3D space, that it is much easier to do so in a one-dimensional (1D) space, as noted above. Then, the 1D space could make it possible to graph the constraint values via the 1D coordinate, so as to identify the set of constraints in conflict and to affect a modification.

Still referring to FIG. 3A, method 300 begins with step 330 that converts the dose of radiation into the curve of irradiation in the body of the patient as a function of the distance to a border of the tumor, a border of the OAR, or both, to generate a graphical representation to identify the dose constraints in violation. We discovered that by making it possible to graph the constraint values via the 1D coordinate system, we are able to visualize the constraint conflict. In other words, we plot the OAR constraints verses distance to a tumor boundary and tumor constraints verses the distance to an OAR boundary on the same graph. After plotting the OAR constraints and the tumor constraints on the same graph, we are able to construct a user interface, i.e. a slider or control point, that causes a shift of the characteristic of the curve along the distance axis and a corresponding localized change to the tumor and OAR constraints.

Step 335 of FIG. 3A includes shifting the curve of irradiation along an axis of the distance to manipulate the graphical representation of the dose constraints in violation, via a user through a user interface connected with the processor. Wherein the user provides a user selected shifted curve of irradiation, in part, via assistance in evaluating the dose constraints in violation through the graphical display, to obtain a shifted curve of irradiation. In particular, we are able to construct systems and methods that are able to make minimal adjustments to the constraints as necessary to obtain a feasible optimization problem, as well as, generate a graphical representation illustrating simultaneously the constraints in conflict.

Specifically, we are able to simultaneously illustrate visually, i.e. by shifting the curve of irradiation along the axis of the distance, the effects of the specific dose of radiation to the tumor, along with the corresponding damaging effects to the surrounding healthy tissue and OARs for the specific radiation dose. By providing a visual display to the user, the user now has the ability to have explicit local control of constraint trade-offs, i.e., parts of OAR that are overdosed via the slider. For example, the user could be a dosimetrist, doctor or person associated with determining dosing or medical issues for the patient. Wherein the user, via the slider of the present disclosure, will have the ability to visually see each potential radiation dose and the corresponding damaging effects to the OARs, and after having reviewed all the possible radiation dosing options, make an informed dosing radiation decision necessary to obtain a constraint set or specific dose of radiation for the patient, i.e. the new position of the shifted curve of irradiation along the axis of the distance. The constraint set may eventually be later decided to be a feasible constraint set to be used to obtain a modified dose of radiation.

Still referring to FIG. 3A, the constraint set may be determined by one of a user, a user having medical knowledge, i.e. a dosimetrist, a feasible constraint optimization algorithm, or some combination thereof. Wherein the feasible constraint set can be based upon determining a compromise between an amount of tumor control versus an amount of damage control to the at least one OAR by the dose of radiation. In other words, a user will need to make a compromise between: (1) sparing the OAR, but under dosing the tumor; (2) adequately irradiating the tumor, but overdosing some or all of an OAR; or (3) some tradeoff between the two. In part, the slider in combination with the features of the present disclosure provides a user to make informed radiation dosing decisions, as well as have an increased accuracy in understanding how the proposed dosing of radiation will have on the tumor and OARs of the patient.

Step 341 includes modifying a portion of at least one dose constraint violated by the shifted curve of irradiation to follow the shifted curve, to identify a modified tumor dose constraint and a corresponding modified OAR dose constraint from a location of the shifted curve along the axis of the distance.

Step 345 of method 300 includes changing the dose of radiation according to the modified dose constraints to a modified dose of radiation.

Step 350 of method 300 includes managing the radiation treatment planning system, according to the modified dose of radiation.

FIG. 3B is a schematic illustrating the method 300 of FIG. 3A that includes processor 340AA in communication with the diagnostic system 150 and with the radiation implementation system 180, as similar to method 100 of FIG. 1B. Also, the diagnostic system 150 generates empirical data of the patient or body 109 positioned on table 108.

The processor 340AA initiates step 330 of method 300 by converting the dose of radiation determined via method 100 into a curve of irradiation in the body of the patient as a function of a distance to a border of the tumor, a border of the OAR, or both, to generate a graphical representation to identify the dose constraints in violation. Wherein upon converting the dose of radiation into the curve of irradiation, shifting the curve of irradiation along an axis of the distance to manipulate a graphical representation of the dose constraints in violation, via a user through a user interface connected with the processor.

Block 335A of FIG. 3B, i.e. step 335 of FIG. 3A, can be accomplished by a user shifting the curve of irradiation along an axis of the distance to manipulate the graphical representation of the constraints in violation, using the graphically display on display 362 via a surface of a user interface, such as keys on a keyboard. It is contemplated the user may use voice commands to initiate the shifting of the curve graphically displayed on display 362. For example, for each shift of the curve, the user is able to graphically visualize each modified tumor constraint and a corresponding modified OAR constraint that directly correlates to the effects of the modified tumor constraint.

Block 340BB dose optimization and block 340CC fall-off curve of radiation can be accomplished by the utilizing block 335A. For example, the user is able to simultaneously illustrate visually, i.e. by shifting the curve of irradiation along the axis of the distance, the effects of the specific dose of radiation to the tumor, along with the corresponding damaging effects to the surrounding healthy tissue and OARs for the specific radiation dose. The visual display provides the user an ability to have explicit local control of constraint trade-offs, i.e., parts of OAR that are overdosed via the slider. The slider allows the user to visually see each potential radiation dose and the corresponding damaging effects to the OARs, and after having reviewed all the possible radiation dosing options, make an informed dosing radiation decision necessary to obtain a constraint set or specific dose of radiation for the patient, i.e. the new position of the shifted curve of irradiation along the axis of the distance. In essence, the user is able to optimize the dose of radiation 340BB via assistance of viewing the fall-off curve of radiation 340CC. Wherein a constraint set may eventually be later decided to be a feasible constraint set used to obtain a modified dose of radiation.

FIG. 3C is a block diagram illustrating a method 380 processed by processor 340DD that initially converts an acquired initial dose of radiation into a curve of irradiation in the body of the patient as a function of the distance to the boundary of the tumor, the boundary of the OAR, or both, to generate a graphical representation to identify the dose constraints in violation.

Step 382 of FIG. 3C includes determining an initial dose of radiation violating constraints.

Step 384 of FIG. 3C includes transforming the initial dose into a curve of radiation as a function of a distance between tumor and OAR.

Step 386 of FIG. 3C includes shifting curve of irradiation along an axis of the distance to manipulate the graphical representation of the dose constraints in violation, via a user through a user interface connected with the processor, to determine a shifted curve of irradiation.

Step 388 of FIG. 3C includes modifying a portion of at least one dose constraint violated by the shifted curve of irradiation to follow the shifted curve, to identify a tumor dose constraint and a corresponding OAR dose constraint from a location of the shifted curve along the axis of the distance.

Step 390 of FIG. 3C includes changing the initial dose of radiation according to the dose constraints to a dose of radiation.

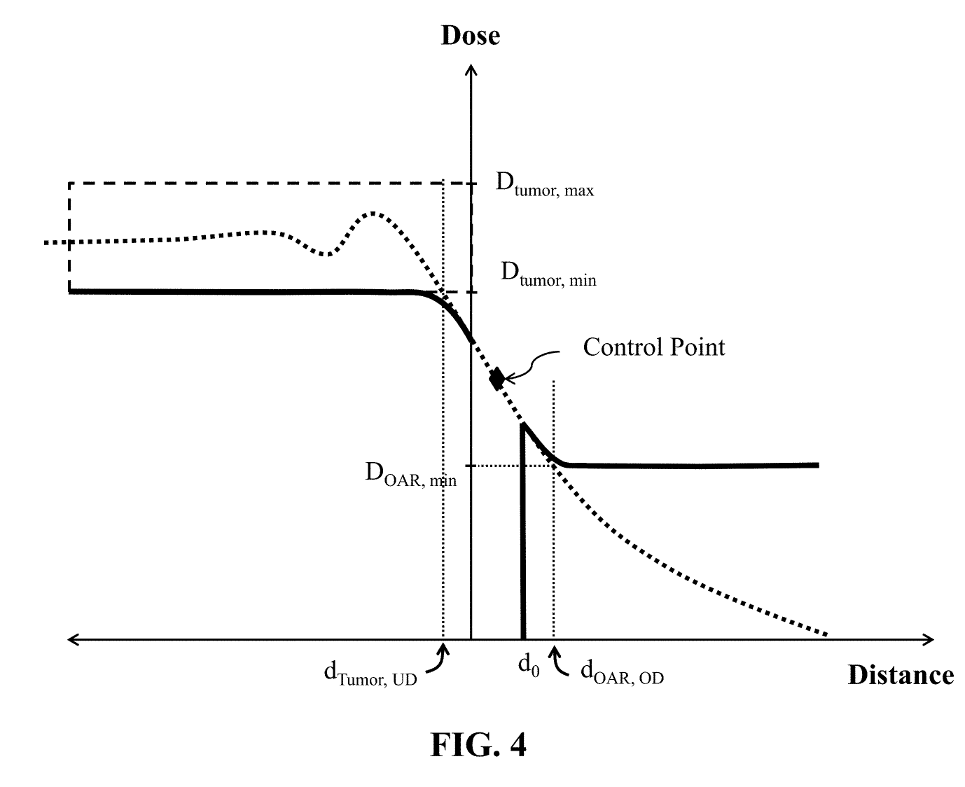

FIG. 4 is a graph illustrating the tumor and OAR constraints modified, i.e. increased in the OAR constraint and decreased in the tumor constraint, as a consequence of the user's or dosimetrist's action in setting the location of the control point at a point intermediate between the two constraint sets. Wherein, the problem being solved according aspects of the present disclosure is the visualization and modification of the set of constraints on the tumor and OARs that are in conflict. FIG. 4 displays a line graph that includes a y-axis corresponding to the dose of radiation and an x-axis corresponding to the distance.

The minimum OAR constraint is shown as D_{OAR, min}, and the position of the OAR boundary along the axes of the distance is shown as d_{OAR, OD}. Further still, the minimum distance between the tumor and the OAR is shown as d₀. The position of the Tumor boundary along the axes of distance is shown as d_{Tumor, UD}. It is noted that “UD” means under dose and “OD” means over dose, such that under and over refer to the original constraint values.]

Because, it is not uncommon to have an inherent conflict between the desire to have high dose in the tumor and low dose in a nearby OAR. Radiation planning is usually an iterative process that involves repeated adjustments of the distribution and intensity of radiation sources, and repeated evaluation of the quality of the plan during the iterations. There are many complications in radiation planning that need to be considered, before implementing a specific radiation plan for a patient.

For example, determining a dose of radiation for a patient is further complicated, due to the technological limitations of radiation beams not being able to concisely target tumor and OAR areas. Sources of radiation are not able to produce a very sharp spatial change in the radiation distribution. For example, a beam of protons may be used in radiation therapy. The beam originates from a particle accelerator such as a synchrotron or cyclotron. As the beam emerges from the accelerator its spatial distribution in the direction transverse to its propagation direction is well approximated by a Gaussian whose size, as determined by its full width at half maximum, is often in the range of a few millimeters up to 15 mm. Additionally, the physics of the propagation of an ion beam in matter is such that it gives rise to a characteristic radiation distribution in its propagation direction known as a Bragg curve. The key feature of the Bragg curve is that the amount of dose deposited as a function of depth inside the matter has a sharp peak (the Bragg peak) at a distance inside the matter that is determined by the energy of the particles, as well as by the composition of the matter through which it propagates. Increasing the energy of the ions increases the depth of the Bragg peak. Likewise increasing the density of the matter reduces the depth of the Bragg peak.

The Bragg peak will occupy a volume of a few 10s of cubic millimeters, usually much smaller than the volume of the tumor. To treat the entire volume of the tumor it is necessary to irradiate it with many Bragg peaks arranged over the tumor volume. This can be achieved, for example, by scanning the particle beam in the direction perpendicular to its propagation direction with electromagnets. Likewise, the Bragg peak can be scanned in its propagation direction by changing the proton energy. During scanning the beam dwells at a multitude of individual locations in the transverse direction for varying times with varying energy such that the radiation dose from the many individual spots combines to produce a large volume of high radiation.

The task of determining the dwelling times for the beam scanning is called dose optimization. Mathematically we can compute the dose deposited by a set of N beams using the expression Dᵢ=Σⱼ₌₀^{N}Aᵢⱼwⱼ, where Dᵢ is the dose to CT voxel i, Aᵢⱼ is a beam matrix that records the amount of dose deposited in CT voxel i by beam j, and wⱼ is the weight, related to the dwelling time, for beam j. The total dose D_{T} is obtained by summing the dose from all voxels, V, by D_{T}(w)=Σᵢ₌₀^{V} Dᵢ.

During the dose optimization we seek to determine a set of values of the beam weights, wⱼ, such that the total dose is minimized subject to a set of constraints on some of the CT voxels. In particular, for tumor voxels the dose is constrained to lie within an acceptable range of values D_{tumor, min}<=Dₜᵤₘₒᵣ<D_{tumor, max} sufficient to kill the tumor cells, while for OAR voxels the dose is constrained to lie below a maximum value, D_{OAR}<D_{OAR, max} which protects the OAR from permanent damage by the radiation. The optimization problem is stated mathematically as

argmin_{w}D_{T}(w)s.t.D_{tumor,min}≦Dₜᵤₘₒᵣ<D_{tumor,max} and D_{OAR}<D_{OAR,max}

where the optimization algorithm iteratively modifies the weights, w, so as to minimizes the total radiation while satisfying the dose constraints on the tumor and OARs. Such an optimization algorithm for performing the dose optimization is Gradient Descent.

Procedurally, the doctor will determine the initial values of the constraints for every tumor and OAR voxel. Then a first dose optimization computation is performed using the dose optimization algorithm that iteratively adjusts the beam weights to attempt to satisfy constraints on the tumor and OAR voxels. However, because the methods and systems of the present disclosure provide for the distance fields of the tumor and the set of OARs to be combined with computed dose distribution or computed dose optimization algorithms, to form a user interface for visualization and control of constraint tradeoffs between the tumor and OARs. The methods and systems of the present disclosure provide doctors with a more informative and optimized approach for determining doses of radiation for patients, along with improving managing the radiation treatment planning system, among other things.

Another complication in determining the set of dose constraints, is that tumor growth is often very rapid with the consequence that the blood supply to the inner regions of the tumor is poor. A poor blood supply in a region of the tumor results in reduced oxygen levels to that region. The reduced oxygen levels can have the biology effect of causing tumor cells to have increased resistance to radiation. Therefore, to improve the tumor eradication by radiation therapy it is needed to increase the dose of radiation to inner parts of the tumor by increasing the corresponding dose constraints. Again, the methods and systems of the present disclosure provide doctors with a more informative and optimized approach in evaluating and controlling the tradeoff between dose constraints that are in conflict, when determining the set of dose constraints to be administered to the patient, among other things.

Still further, another complication it is not uncommon for the distance between the tumor and the OARs to be less than the size of the proton beam. Therefore, in general it is impossible to simultaneously satisfy both the tumor dose constraints and the OAR dose constraints. A radiation distribution that satisfies the dose constraints within the tumor can often produce a dose that is too high in a nearby OAR. Likewise producing a low dose in an OAR will most likely result in a low dose on the parts of the tumor near the OAR. In such a situation the optimization problem is known as “infeasible” as there exists no set of weights, w, which can simultaneously satisfy all of the tumor and OAR constraints. In other words, as noted above, the methods and systems of the present disclosure provide doctors with a more informative and optimized approach in evaluating and controlling the tradeoff between dose constraints that are in conflict, when determining the set of dose constraints to be administered to the patient, among other things.

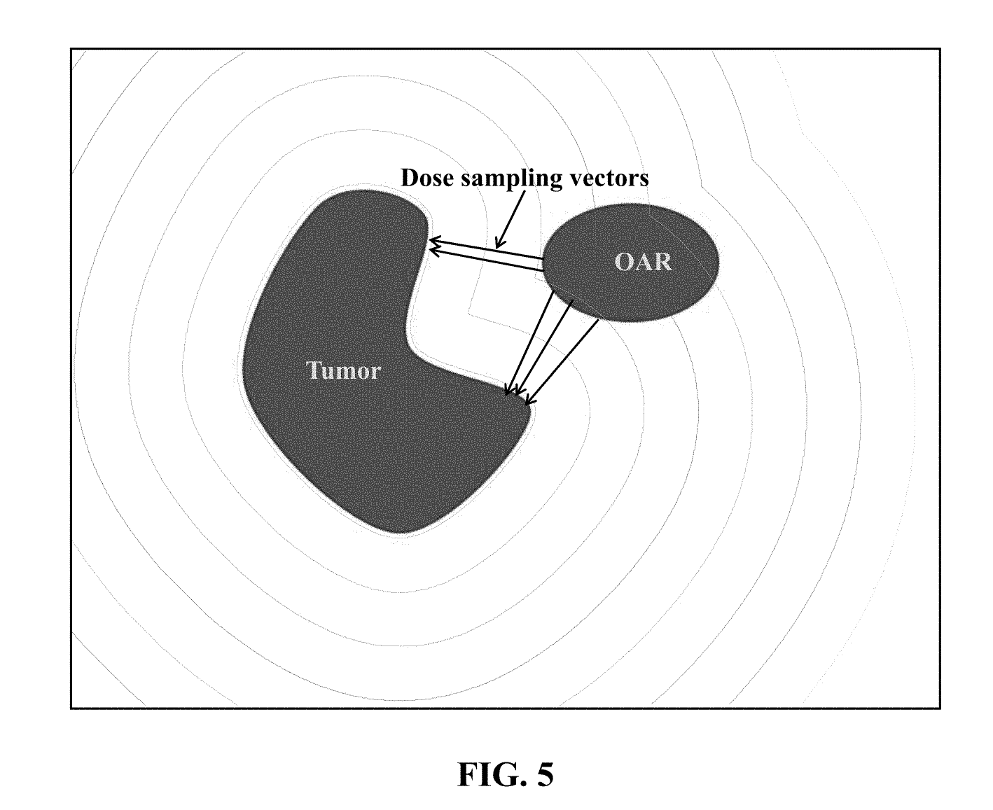

FIG. 5 is a schematic illustrating a set of dose fall-off curves that can be obtained by sampling a computed radiation dose from a set of points on the OAR along a set of distance sampling vectors, according to embodiments of the present disclosure.

According to aspects of the present disclosure, the modification of constraints begins after determining an initial dose optimization from determining the constraint set, i.e. the set of unsatisfied constraints, as well as the 3D dose distribution. From the dose distribution, the dose fall-off curve can be obtained by a number of methods. FIG. 5 shows a method to obtain a dose fall-curve, such that a set of dose fall-off curves can be obtained along a set of directions that originate from a set of points on the OAR boundary and follow dose sampling vectors that may, for example, be the distance field gradient vectors. FIG. 5 further illustrates five such dose sample vectors from the OAR to the tumor. The set of dose curves obtained along the dose sampling vectors can be compared to one another to determine the dose curve that has the slowest rate of dose fall-off, i.e. is the worst case. This worst case curve can then be used as the characteristic dose fall-off curve. From this set of dose curves, a characteristic dose fall-off curve can be obtained by a number of methods such as averaging.

Still referring to FIG. 5, another method for obtaining the dose fall-off curves may be from using the slider, noted above. For example, in order to obtain the dose fall-off curves, a constraint set first needs to be determined. The constraint set can be determined, in part, using the slider by a user that can visual display explicit local control of constraint trade-offs, i.e., parts of OAR that are overdosed via the slider. Wherein, the slider allows the user to visually see each potential radiation dose and the corresponding damaging effects to the OARs, along with viewing each dose fall-off curve of radiation. In essence, the user is able to review all the possible radiation dosing options, make an informed dosing radiation decision necessary to obtain a constraint set or optimize the dose of radiation via assistance of viewing all the possible fall-off curves of radiation.

Still referring to FIG. 5, alternately, the dose curves obtained by the previous method can be averaged together to form an average dose curve. This average curve can then be used as the characteristic dose fall-off curve.

Alternately, a single characteristic dose fall-off curve can be created by taking the maximum dose value from the set of dose curves at each distance. The characteristic dose fall-off curve is then the worst case at every distance from the tumor.

Alternately, the spatial profile characteristic of the radiation source may be used as the characteristic dose fall-off curve. For example, an ion beam has a transverse profile, often modelled as a Gaussian, over which the dose falls from its maximum value to zero. If the maximum is scaled so that its value matches the mean value of the minimum and maximum tumor dose constraints it can then be used as the characteristic dose fall-off curve.

Still referring to FIG. 5, a further augmentation to the visualization provided by the present disclosure is obtained by adding to the plot a curve showing the overlap volume histogram (OVH) as described by U.S. Pat. No. 8,688,618 B2. As illustrated in FIG. 5, the OVH is a graph that indicates the fraction of the OAR that is within a given distance of the tumor boundary. It may be that the OAR geometry is such that the fraction of the OAR voxels whose dose exceeds the desired maximum dose is small (curve B). In this case only a small part of the OAR is affected by overdose and, therefore, a small part of the OAR will be sacrificed in order to adequately dose the adjacent tumor voxels. Alternately, the OAR geometry may be such that a large fraction of it is close to the tumor (curve A) and will therefore be affected by overdose. In this case the dosimetrist may prefer to more strongly protect the OAR so as to preserve its functionality and allow a corresponding under dose in adjacent tumor voxels.

Referring to FIG. 5, it is possible that altering the constraint set on one section of the tumor to protect a first OAR may have a negative effect on the dose to a second OAR. This is possible because the optimization algorithm may attempt to compensate for a reduction in dose in one part of the tumor by an increase in dose in another part. Therefore, a useful extension of the present disclosure is to display multiple graphs on the same form, each associated with a different OAR, so that effect of a change in constraints for one OAR on the dose for other OARs may be observed thereby enabling a higher level of tradeoff between OARs. For example, it may be acceptable to increase the dose constraints to a first OAR in order to improve the protection for a second, more important, OAR.

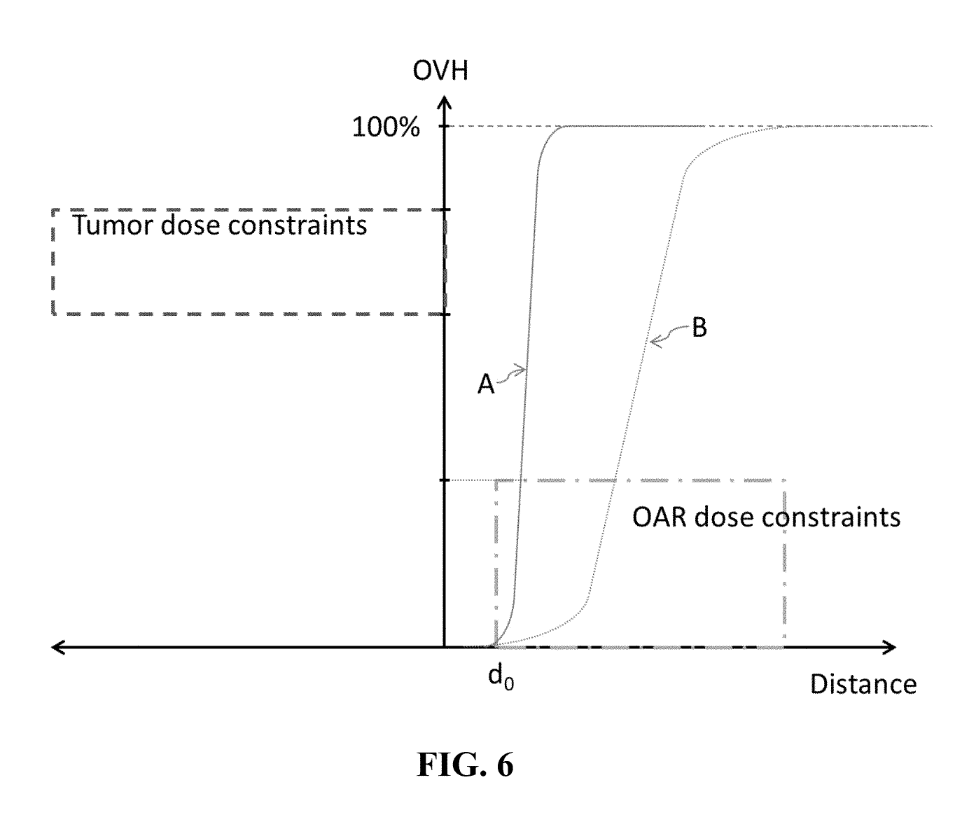

FIG. 6 is a graph illustrating an overlap volume histogram (OVH) of the OAR indicating a fraction of the OAR voxels within a given distance of the tumor, according to embodiments of the present disclosure. For example, curve A illustrates an OAR that has a large fraction of its voxels close to the tumor, while curve B illustrates an OAR whose geometry is such that fewer voxels are close to the tumor.

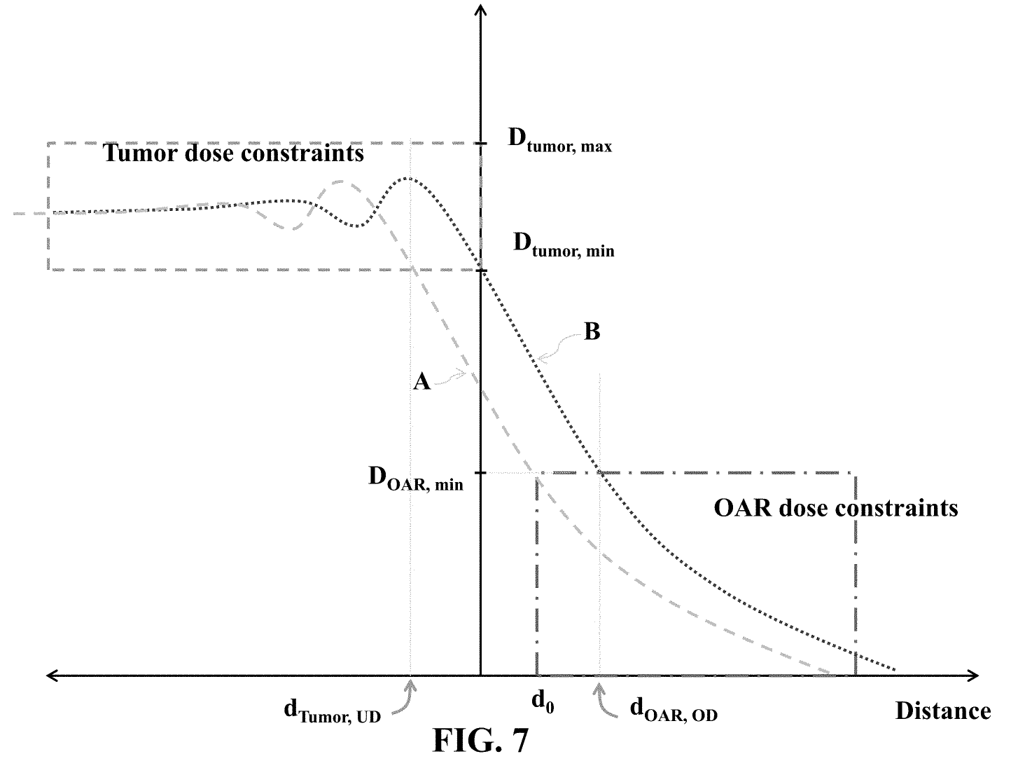

FIG. 7 is a graph illustrating a long dashed box on the left showing the tumor minimum and maximum dose constraints, the dot-dashed box on the right showing the OAR maximum dose constraints, and curves A and B that are two characteristic dose curves whose fall-off is the fastest achievable due to limitations in the beam size and tissue heterogeneity, according to embodiments of the present disclosure.

For example, the graph of FIG. 7 shows the tumor dose constraints (D_{tumor, min} and D_{tumor, max}) vs. d₀−d_{OAR} (long dashed box), the OAR dose constraints (D_{OAR, max}) vs d_{T} (dashed box), and two characteristic dose fall-off curves A and B, according to embodiments of the present disclosure.

If we consider the spatial relationship between the tumor boundary and an OAR boundary, we can observe that there is a minimum distance of closest approach between the two sets, hereafter referred to a do. Furthermore, the distance from the tumor boundary d_{T} can be related to the distance from the OAR boundary d_{OAR} by d_{T}=d₀−d_{OAR}.

Still referring to FIG. 7, the constraint conflicts can be visualized by plotting to a single graph, whose vertical axis is dose and whose horizontal axis is distance, two sets of data:
1) The values of the OAR constraints as a function of their distance from the tumor boundary;
2) The values of the tumor constraints as a function of do minus the distance from the OAR boundary; and
3) A characteristic dose fall-off curve.

In particular, the graph of FIG. 7 shows the tumor minimum and maximum dose constraints, the dot-dashed box on the right showing the OAR maximum dose constraints, and A and B are two characteristic dose curves whose fall-off is the fastest achievable due to limitations in the beam size and tissue heterogeneity, as noted above. Wherein, curve A satisfies the OAR constraints, but under-doses the set of tumor voxels having d>d_{Tumor, UD}. Curve B satisfies the tumor constraints, but overdoses the OAR voxels having d<d_{OAR, OD}.

As FIG. 4 demonstrated aspects of the present disclosure of modifying the constraints to obtain a feasible optimization problem. As stated earlier, the optimization problem is fundamentally infeasible since there are no sets of beam weights that can satisfy all constraints. Therefore, the dosimetrist is required to make a compromise between (a) sparing the OAR, but under dosing the tumor, (b) adequately irradiating the tumor, but overdosing some or all of an OAR, or (c) some tradeoff between the two.

Therefore, a user interface can be constructed by placing a control point in the middle of the characteristic dose curve that the dosimetrist can move along the distance axis to choose the degree to which they favor either the tumor or the OAR. As a result of the dosimetrist's selection the tumor and OAR constraints are modified such that they are given values slightly greater than that of the characteristic dose curve. This implies that those voxels whose distance is closest to the opposing set have the greatest change in their constraint values.

Still referring to FIG. 7, to implement the aspects of the present disclosure it is necessary to be able to first determine the Euclidean distances, known as a distance field, from the boundaries of the tumor and OARs. This can be performed using the algorithm called a Euclidean distance transform. The input to the distance transform for a given structure (i.e., tumor or OAR) is a binary 3D voxel data array where each voxel that is within the structure is given a value of 1, and all other voxels are given values of 0. The distance transform algorithm then returns a new co-located 3D data voxel array wherein the floating point value of each voxel is the minimum Euclidean distance from the same voxel of the input voxel array to the boundary of the structure. It is then possible to determine for a given voxel of the opposite category (OAR or tumor) the distance to the opposing boundary.

A numerical gradient vector for the distance field is straightforward to compute by a number of standard numerical differentiation algorithms such as finite differences. The gradient vector of a distance field at a point p has a direction that points from p toward a point on the boundary that has the minimum distance from p to the boundary; it is the direction to go that gets to the boundary in the least distance. The gradient vectors are always perpendicular to the distance isocontours illustrated in FIG. 5.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine. Typically, the functionality of the program modules may be combined or distributed as desired in various embodiments.

Also, the embodiments of the present disclosure may be embodied as a method, of which an example has been provided. The acts performed as part of the method may be ordered in any suitable way. Accordingly, embodiments may be constructed in which acts are performed in an order different than illustrated, which may include performing some acts concurrently, even though shown as sequential acts in illustrative embodiments. Further, use of ordinal terms such as “first,” “second,” in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Although the present disclosure has been described with reference to certain preferred embodiments, it is to be understood that various other adaptations and modifications can be made within the spirit and scope of the present disclosure. Therefore, it is the aspect of the append claims to cover all such variations and modifications as come within the true spirit and scope of the present disclosure.

What is claimed is:

1. A radiation treatment method for determining a dose of radiation, comprising:
determining, for a tumor voxel in a set of tumor voxels from stored data, a value of a maximum dose constraint and a minimum dose constraint, for treating the tumor voxel based on a distance function of at least one distance field of the tumor and distance fields of organs at risk (OARs), wherein each dose constraint on each tumor voxel is a function of a distance from the tumor voxel to a boundary of the tumor, and distances to boundaries of OARs;
determining, for an OAR voxel in a set of OAR voxels in an OAR of interest of the OARs, a value of a maximum dose constraint based on the at least one distance field of the tumor, wherein each dose constraint on each OAR voxel is a function of a distance from the OAR voxel to the boundary of the tumor;
determining a tumor dose constraint and a corresponding OAR dose constraint to obtain a dose constraint set according to a threshold dose constraint set; and
determine the dose of radiation, according to the dose constraint set, wherein the dose of radiation is used for managing the radiation treatment planning system.

2. The method of claim 1, wherein the threshold dose constraint set includes a user selected threshold dose constraint set.

3. The method of claim 2, wherein the user selected threshold dose constraint set is based on, one or a combination of, the determined dose constraints, a compromise between an amount of tumor control of a specific tumor dose constraint versus an amount of damage control to a OAR voxel of a specific corresponding OAR dose constraint and stored historical radiation dose treatments for tumor types, tumor voxels and OARS similar to the set of tumor voxels and OARs from stored data.

4. The method of claim 1, wherein the function affecting the tumor dose constraints depends additionally on the dose of radiation to the OARs, and the function affecting the OAR radiation dose constraints depends additionally on the dose of radiation to the tumor.

5. The method of claim 1, wherein the distance function is a linear combination algorithm or a non-linear combination algorithm, of a tumor distance field and the OAR distance field, and the distance function has parameters that control a shape of the distance function.

6. The method of claim 5, wherein the parameters include k, D_{tumor, min}^{max} control a slope and a maximum dose limit, respectively.

7. The method of claim 5, wherein the parameters of the distance function are controlled by a user through a user interface in communication with at least one processor.

8. The method of claim 7, wherein the user interface is graphical.

9. The method of claim 1, further comprising:
converting the dose of radiation into a curve of irradiation in a body of a patient as a function of a distance to a border of the tumor, a border of the OAR, or both, to generate a graphical representation to identify the dose constraints in violation;
shifting the curve of irradiation along an axis of the distance to manipulate the graphical representation of the dose constraints in violation, via a user through a user interface connected with the at least one processor, to determine a shifted curve of irradiation according to a threshold dose constraint set, in part, via assistance evaluating the dose constraints in violation through the graphical display;
modifying a portion of at least one dose constraint violated by the shifted curve of irradiation to follow the shifted curve, to identify a modified tumor dose constraint and a corresponding modified OAR dose constraint from a location of the shifted curve along the axis of the distance;
changing the dose of radiation according to the modified dose constraints to a modified dose of radiation; and
managing the radiation treatment planning system, according to the modified dose of radiation.

10. The method of claim 1, further comprising:
at least one processor and at least one non-transitory storage memory having the stored data and computer readable instructions executable by the at least one processor, wherein the execution of the computer readable instructions by the at least one processor.

11. The system of claim 10, wherein the stored data specific to the set of tumor voxels includes three-dimensional data (3D) patient imaging data transformed into two-dimensional (2D) patient imaging data, and a graphical user interface connected to the at least one processor displays a (2D) line graph that includes a y-axis corresponding to the dose of radiation and an x-axis corresponding to the distance.

12. The method of claim 10, further comprising:
converting, an acquired initial dose of radiation by the at least one processor, into a curve of irradiation in a body of a patient as a function of the distance to the boundary of the tumor, the boundary of the OAR, or both, to generate a graphical representation to identify the dose constraints in violation;
shifting the curve of irradiation along an axis of the distance to manipulate the graphical representation of the dose constraints in violation, via a user through a user interface connected with the at least one processor, to determine a shifted curve of irradiation according to a threshold dose constraint set, in part, via assistance evaluating the dose constraints in violation through the graphical display;
modifying a portion of at least one dose constraint violated by the shifted curve of irradiation to follow the shifted curve, to identify a tumor dose constraint and a corresponding OAR dose constraint from a location of the shifted curve along the axis of the distance;
changing the initial dose of radiation according to the dose constraints to a dose of radiation; and
managing the radiation treatment planning system, according to the dose of radiation.

13. The method of claim 12, the user determines the user selected threshold dose constraint set based on, one or a combination of, the determined dose constraints, a compromise between an amount of tumor control of a specific tumor dose constraint versus an amount of damage control to a OAR voxel of a specific corresponding OAR dose constraint and stored historical radiation dose treatments for tumor types, tumor voxels and OARS similar to the set of tumor voxels and OARs from stored data.

14. A radiation treatment planning system for determining a dose of radiation, comprising:
at least one processor; and
at least one non-transitory storage memory having stored data and computer readable instructions executable by the at least one processor, wherein the execution of the computer readable instructions by the at least one processor is configured to:
determine, for a tumor voxel in a set of tumor voxels from patient imaging data of the stored data, a value of a maximum dose constraint and a minimum dose constraint, for treating the tumor voxel based on a distance function of at least one distance field of the tumor and distance fields of organs at risk (OARs) in a set of OARs, wherein each dose constraint on each tumor voxel is a function of a distance from the tumor voxel to a boundary of the tumor, and distances to boundaries of OARs in the set of OARs;
determine, for an OAR voxel in a set of OAR voxels in an OAR of interest from the set of OARs, a value of a maximum dose constraint based on the at least one distance field of the tumor, wherein each dose constraint on each OAR voxel is a function of a distance from the OAR voxel to the boundary of the tumor;
determining a tumor dose constraint and a corresponding OAR dose constraint to obtain a dose constraint set according to a threshold dose constraint set; and
determine the dose of radiation, according to the dose constraint set, wherein the dose of radiation is used for managing the radiation treatment planning system.

15. The system of claim 14, wherein the threshold dose constraint set includes a user selected threshold dose constraint set, such that the user selected threshold dose constraint set is based on, one or a combination of, the determined dose constraints, a compromise between an amount of tumor control of a specific tumor dose constraint versus an amount of damage control to a OAR voxel of a specific corresponding OAR dose constraint and stored historical radiation dose treatments for tumor types, tumor voxels and OARS similar to the set of tumor voxels and OARs from stored data.

16. The system of claim 14, wherein the function affecting the tumor dose constraints depends additionally on the dose of radiation to the OARs, and the function affecting the OAR radiation dose constraints depends additionally on the dose of radiation to the tumor.

17. The system of claim 14, wherein the distance function is a linear combination algorithm or a non-linear combination algorithm, of a tumor distance field and the OAR distance field, and the distance function has parameters that control a shape of the distance function.

18. The system of claim 17, wherein the parameters of the distance function are controlled by a user through a user graphical interface in communication with at least one processor, wherein the parameters include

19. The system of claim 14, further comprising:
convert the dose of radiation into a curve of irradiation in a body of a patient as a function of a distance to a border of the tumor, a border of the OAR, or both, to generate a graphical representation to identify the dose constraints in violation;
shift the curve of irradiation along an axis of the distance to manipulate the graphical representation of the dose constraints in violation, via a user through a user interface connected with the at least one processor, wherein the user provides a user selected shifted curve of irradiation, in part, via assistance evaluating the dose constraints in violation through the graphical display, to obtain a shifted curve of irradiation;
modify a portion of at least one dose constraint violated by the shifted curve of irradiation to follow the shifted curve, to identify a modified tumor dose constraint and a corresponding modified OAR dose constraint from a location of the shifted curve along the axis of the distance;
change the dose of radiation according to the modified dose constraints to a modified dose of radiation; and
manage the radiation treatment planning system, according to the modified dose of radiation.

20. The system of claim 14, further comprising:
convert, an acquired initial dose of radiation by the at least one processor, into a curve of irradiation in a body of a patient as a function of the distance to the boundary of the tumor, the boundary of the OAR, or both, to generate a graphical representation to identify the dose constraints in violation;
shift the curve of irradiation along an axis of the distance to manipulate the graphical representation of the dose constraints in violation, via a user through a user interface connected with the at least one processor, wherein the user provides a user selected shifted curve of irradiation, in part, via assistance evaluating the dose constraints in violation through the graphical display, to obtain a shifted curve of irradiation;
modify a portion of at least one dose constraint violated by the shifted curve of irradiation to follow the shifted curve, to identify a modified tumor dose constraint and a corresponding modified OAR dose constraint from a location of the shifted curve along the axis of the distance;
change the dose of radiation according to the modified dose constraints to a modified dose of radiation; and
manage the radiation treatment planning system, according to the modified dose of radiation.
